(19) United States
(12) Patent Application Publication
(10) Pub. No.: US 2023/0414954 A1
(43) Pub. Date: Dec. 28, 2023

(54) PASSIVE WIRELESS MULTI-CHANNEL IMPLANTABLE DEVICE

(71) Applicants: Jennifer BLAIN CHRISTEN, Chandler, AZ (US); Shiyi LIU, Tempe, AZ (US); Ahmed Abed BENBUK, Tempe, AZ (US); Daniel GULICK, Tempe, AZ (US)

(72) Inventors: Jennifer BLAIN CHRISTEN, Chandler, AZ (US); Shiyi LIU, Tempe, AZ (US); Ahmed Abed BENBUK, Tempe, AZ (US); Daniel GULICK, Tempe, AZ (US)

(73) Assignee: ARIZONA BOARD OF REGENTS ON BEHALF OF ARIZONA STATE UNIVERSITY, Scottsdale, AZ (US)

(21) Appl. No.: 18/342,688

(22) Filed: Jun. 27, 2023

Related U.S. Application Data
(60) Provisional application No. 63/356,471, filed on Jun. 28, 2022.

(51) Int. Cl. A61N 1/378; H02J 50/20

(52) U.S. Cl. CPC A61N 1/3787; H02J 50/20

(57) ABSTRACT

A wireless multi-channel implantable device. In some embodiments, the system includes: a wireless reception element, for receiving power wirelessly through tissue of a subject; a power management and storage circuit, for storing a portion of the received power; and a control circuit for controlling the delivery of current from the power management and storage circuit to each of a plurality of stimulation electrodes individually, based on a modulation of the received power.

### CROSS-REFERENCE TO RELATED APPLICATION(S)

The present application claims priority to and the benefit of U.S. Provisional Application No. 63/356,471, filed Jun. 28, 2022, entitled “PASSIVE WIRELESS MULTI-CHANNEL STIMULATOR”, the entire content of which is incorporated herein by reference.

### FIELD

One or more aspects of embodiments according to the present disclosure relate to implantable devices, and more particularly to a passive wireless multi-channel implantable device.

### BACKGROUND

Passive, wireless stimulators may have advantages over alternative systems due to their ability to provide therapy and rehabilitation by targeting an organ with monophasic or biphasic stimulation pulses. Electrical stimulation may be used, for example, to treat atrial fibrillation, and to repair damaged nerves. Wireless stimulators may offer the advantage of reducing the risk of infection and inflammation by eliminating wires that protrude through the skin to deliver the stimulation signal into the intended tissue. Extending the operation of wireless stimulators from single- to multi-channel enhances the spatial diversity of the wireless stimulator with many useful therapeutic applications.

It is with respect to this general technical environment that aspects of the present disclosure are related.

### SUMMARY

Some embodiments include an ultra-low-power wireless, multi-band stimulator which combines RF energy harvesting and wake-up functionalities for on-demand and selective activation of a specific stimulation channel. The stimulator does not require a battery to operate, and it relies on passive components to harvest energy from the wireless signal. Moreover, it may use a small number of active components, such as an ultra-low power data slicer and a shift register configured as a channel selector, to turn on a stimulation channel upon the reception of a pulse-width-modulated wake-up signal.

In some embodiments, the simplicity of the baseband circuit allows the stimulator to be powered from harvested energy during the stimulation period of one or more specific channels. An external transmitter can be used to power the stimulator circuit by transmitting a continuous signal, and modulation of the carrier transmitted by the external transmitter may be used to convey a wake-up signal to activate a specific stimulation channel.

According to an embodiment of the present disclosure, there is provided a system, including: a wireless reception element, for receiving power wirelessly through tissue of a subject; a power management and storage circuit, for storing a portion of the received power; and a control circuit for controlling the delivery of current from the power management and storage circuit to each of a plurality of stimulation electrodes individually, based on a modulation of the received power.

In some embodiments, the wireless reception element is an antenna.

In some embodiments, the control circuit is configured: to receive a first pulse having a first pulse width and a second pulse having a second pulse width, different from the first pulse width, to convert the first pulse to a first binary value; and to convert the second pulse to a second binary value different from the first binary value.

In some embodiments, the system includes a rectifier for rectifying an alternating current (AC) signal received from the wireless reception element.

In some embodiments, the control circuit includes a passive demodulator circuit for converting the first pulse and the second pulse to the first binary value and the second binary value, respectively.

In some embodiments, the passive demodulator circuit includes a low-pass filter.

In some embodiments, the low-pass filter is a resistor-capacitor (RC) low-pass filter.

In some embodiments, the control circuit includes a shift register, configured: to receive the first binary value and the second binary value; to output the first binary value at a first parallel output of the shift register; and to output the second binary value at a second parallel output of the shift register.

In some embodiments, the system includes a rectifier for rectifying an alternating current (AC) signal received from the wireless reception element, wherein: the control circuit includes a rectifier for rectifying an alternating current (AC) signal received from the wireless reception element; and the shift register has a clock input, connected to the rectifier.

In some embodiments, the clock input is connected to the rectifier through an inverter.

In some embodiments: the first parallel output of the shift register is connected to a first stimulation electrode of the plurality of stimulation electrodes, and the second parallel output of the shift register is connected to a second stimulation electrode of the plurality of stimulation electrodes.

In some embodiments, the first parallel output of the shift register is connected to the first stimulation electrode of the plurality of stimulation electrodes through a switch, the switch being configured to selectively route current from the power management and storage circuit to the first stimulation electrode, in accordance with a control signal received from the first parallel output of the shift register.

In some embodiments, the first parallel output of the shift register is connected to the switch through a low-pass filter.

In some embodiments, the power management and storage circuit includes a first capacitor for supplying the current for the stimulation electrodes.

In some embodiments, the power management and storage circuit includes a direct current to direct current (DC-DC) converter, configured to charge the first capacitor.

In some embodiments, the control circuit includes a shift register, configured to be powered by the DC-DC converter and the first capacitor.

In some embodiments, the system further includes a rectifier and a data slicer, wherein: the control circuit includes a passive demodulator circuit; the rectifier is connected to the wireless reception element; and the data slicer has an input connected to the rectifier, an output connected to the passive demodulator circuit, and a power supply connection connected to the first capacitor.

In some embodiments, the system includes a dielectric or semiconductor substrate supporting the power management and storage circuit and the control circuit, the substrate, the power management and storage circuit and the control circuit together occupying a volume of less than 1,000 cubic millimeters.

In some embodiments, the substrate is substantially flat, and has a thickness of less than 4 mm.

According to an embodiment of the present disclosure, there is provided a system, including: a wireless reception element, for receiving power wirelessly through tissue of a subject; and a shift register, the shift register being configured: to be powered by the wirelessly received power; to have stored in it a data word received as a modulation of the wirelessly received power; and to control delivery of current to a stimulation electrode, of a plurality of stimulation electrodes, based on a bit of the data word.

### BRIEF DESCRIPTION OF THE DRAWINGS

These and other features and advantages of the present disclosure will be appreciated and understood with reference to the specification, claims, and appended drawings wherein:

FIG. 1 is a simplified schematic diagram of a passive wireless multi-channel stimulator, according to an embodiment of the present disclosure;

FIG. 2 is a schematic diagram of a data slicer, according to an embodiment of the present disclosure;

FIG. 3A is a graph of a waveform, according to an embodiment of the present disclosure;

FIG. 3B is a graph of a waveform, according to an embodiment of the present disclosure; and

FIG. 4 is a simplified schematic diagram of a passive wireless multi-channel stimulator, according to an embodiment of the present disclosure.

### DETAILED DESCRIPTION

The detailed description set forth below in connection with the appended drawings is intended as a description of exemplary embodiments of a passive wireless multi-channel stimulator provided in accordance with the present disclosure and is not intended to represent the only forms in which the present disclosure may be constructed or utilized. The description sets forth the features of the present disclosure in connection with the illustrated embodiments. It is to be understood, however, that the same or equivalent functions and structures may be accomplished by different embodiments that are also intended to be encompassed within the scope of the disclosure. As denoted elsewhere herein, like element numbers are intended to indicate like elements or features.

As mentioned above, in some embodiments, a passive multi-channel stimulator relies on energy harvesting and provides selective, on-demand channel activation. The multi-channel stimulator may be implanted into a subject (e.g., a patient) and may be used to provide electrical stimulation to a target organ (e.g., to the heart or to the brain) with spatial and temporal diversity that can be useful for therapeutic purposes. The passive wireless multi-channel stimulator may include a set of components to harvest a wireless signal and store the harvested energy in an energy storage element, and a baseband circuit which demodulates a suitable control signal (e.g., a “wake-up” signal or a reset signal) to activate one or more specific stimulation channels. A passive wireless multi-channel stimulator of some embodiments may be used for stimulation of any part of the body, such as the central or peripheral nervous system, or the heart. In some embodiments, a similar circuit is used to sense electrical signals anywhere within the body.

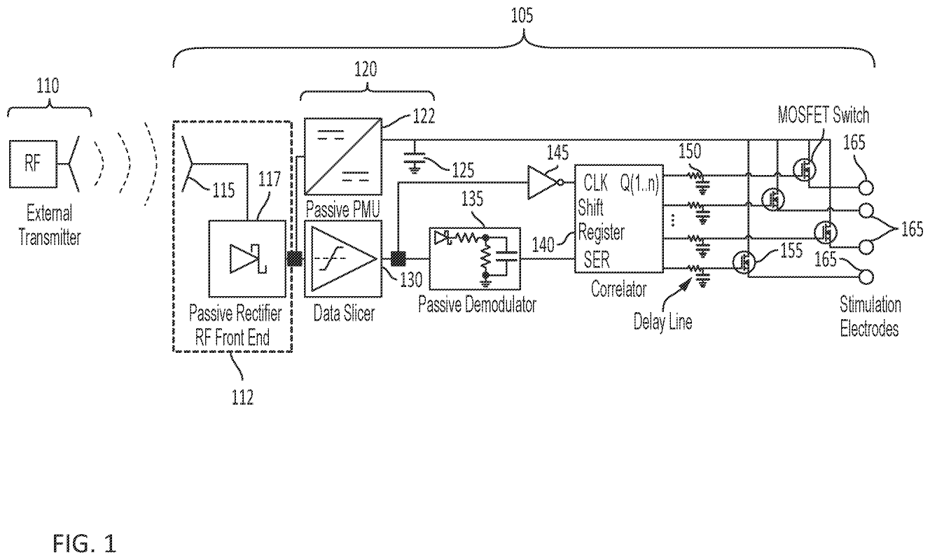

FIG. 1 is a simplified schematic diagram of the passive wireless multi-channel stimulator 105, and of an external transmitter 110, in some embodiments. The stimulator may include a wireless reception element 115 (e.g., an antenna) to receive a wireless signal and a rectifier 117 to rectify the received signal. The antenna may be, for example, a dipole antenna (e.g., a dipole antenna having a total length between 0.12 wavelengths and 0.8 wavelengths, e.g., a quarter-wave dipole antenna, or a half-wave dipole antenna), or a patch antenna (e.g., a stacked patch antenna) or a loop antenna. High dielectric loading or current path shortening may be used to reduce the size of the antenna. High dielectric loading may involve using a substrate (or superstrate) with a high relative dielectric constant (εᵣ) to reduce the effective wavelength (λ_{g}). In some embodiments, Rogers 6010 (which has εᵣ=10.2) is used as a substrate (or superstrate) material. The effective wavelength may be found using λ_{g}=λ_{c}/\sqrt{εᵣ}, where λ_{c} is the wavelength in free space. As such, when a substrate with a high relative dielectric constant is used the antenna may operate at a relatively low frequency, and substrates with a high dielectric constant may be used to reduce the overall size of the antenna. Current path shortening may involve the use of shorting pins on a patch antenna to influence the current path and result in the antenna operating at a lower frequency than an otherwise similar patch antenna without shorting pins.

The received signal (which may be an amplitude modulated alternating current (AC) signal) may be rectified (as discussed in further detail below). The rectified signal may be used to deliver stimulation pulses through a specific channel. Concurrently, the rectified signal may be used to power baseband components that are responsible for the demodulation of the control signal and selective channel activation.

The external transmitter 110 may include a transmitter and a wireless transmission element (e.g., an antenna), and may transmit a modulated control signal (e.g., a wake-up signal) which contains the address or addresses of the target channel or channels to activate a particular stimulation channel. The wireless stimulator 105 contains a digital baseband circuit which includes several components to digitize and demodulate the wake-up signal. A shift register 140 may be employed as a channel selector to activate a stimulation channel and deliver the harvested voltage as a stimulation pulse.

The external transmitter 110 may transmit a radio frequency (RF) signal (e.g., a 2.4 GHz signal) and perform on-off-keying. The external transmitter 110 may be connected to a wireless transmission element (e.g., an antenna) for producing a wireless signal. For example, a signal source (e.g., an Agilent model N5181A analog signal source) may be connected, through a power amplifier (e.g., a Mini-Circuits model ZHL-30 W-252-S+high power amplifier) to a suitable transmitting antenna (e.g., a TDK-HRN-0118 horn antenna, with a gain of 10 dB). The wireless transmission element may be tuned to operate at a specific operating frequency (f0). In some embodiments the wireless reception element is a coil, inductively coupled to the wireless transmission element (which may also be a coil) of the external transmitter 110.

The passive wireless multi-channel stimulator 105 may include an RF front end 112, a power management and storage circuit 120, and a baseband circuit (or “control circuit”) for demodulating control signals from the external transmitter 110 and performing selective channel activation. These components may be responsible for energy harvesting and selective channel activation. The RF front end 112 may include the wireless reception element 115 which receives and converts the wireless signal into an alternating voltage and current, and a rectifier 117, which converts the RF signal at the output of the reception element into a modulated direct current (DC) voltage. As mentioned above, the wireless reception element 115 may be implemented, for example, as a coil or an antenna which is tuned to operate at the operating frequency (f0).

A matching network (e.g., an inductor (L) or a capacitor (C), or an LC circuit (e.g., a parallel LC circuit), or a transmission line matching circuit) may be connected between the wireless reception element 115 and the rectifier 117 to transform the input impedance of the rectifier 117 to that of the wireless reception element 115 at the operating frequency (f0) and minimize the loss in the signal power that is delivered to the rectifier 117.

The design of the matching circuit may depend on the antenna. For example, a single series or shunt lumped element may be used with some antennas; a matching circuit of this kind may be relatively compact. Other types of matching circuits, such as ones including one or more transmission line elements, may be used, at the expense, in some cases, of increased size. The matching circuit is used to match the impedance of the rectifier and the converter circuit 122 to the antenna. For example, the input impedance of the converter circuit 122, if it is a boost converter, may be found using the following equation:

$${R}_{in} = \frac{2 {Lf}_{sw}}{{D}^{2}} ( 1 - \frac{{V}_{in}}{{V}_{0}} )$$

where L is the inductance of the power inductor, f_{sw} is the switching frequency, D is the duty cycle, and Vin and Vo are the input and output voltages, respectively. This equation shows that the input impedance is independent of the DC load resistance and input RF frequency. Therefore, a boost converter may be used to obtain a wide load bandwidth regardless of DC load resistance and RF frequency.

The rectifier may be a half-wave rectifier (e.g. a single Schottky diode, such as a Toshiba JDH2S02SL Schottky diode), or, for example, a transistor-based-rectifier (e.g., a diode-connected field effect transistor), a Dickson voltage multiplier, or a full-wave rectifier. One or more Schottky diodes may be used (instead of, e.g., PN diodes) to reduce loss in the rectifier.

The power management and storage circuit 120 may boost and regulate the rectifier's DC output voltage and operate as a power supply for powering some of the components of the baseband circuit. The power management and storage circuit 120 may include a converter circuit 122 (e.g., a DC-DC converter) and a storage capacitor 125. The converter circuit 122 may be implemented using a boost or buck converter (such as a Texas Instruments bq25570 nano power boost charger and buck converter) and may include a voltage regulator. The storage capacitor 125 may operate as an energy storage element for biasing the components (e.g., the data slicer 130, the inverter 145 and the shift register 140, discussed in further detail below) connected to a DC power source. The storage capacitor 125 may have a capacitance of about (e.g., within 50% of) 100 microFarads. In some embodiments, a battery, another electrochemical storage element, or a supercapacitor may be used as an energy storage element, instead of, or in addition to, the storage capacitor 125. The storage capacitor 125 may also supply current to the stimulation electrodes 165.

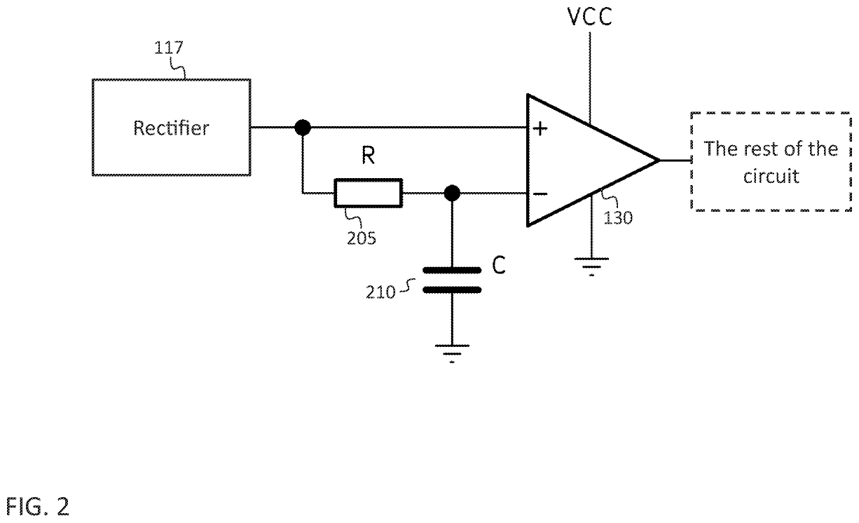

As mentioned above, the baseband circuit may demodulate control signals from the external transmitter 110 and perform selective channel activation. It may include a data slicer 130 (e.g., a Texas Instruments TLV3691 nanopower comparator), a passive demodulator 135, an inverter 145, a shift register 140 (e.g., a Texas Instruments SN74HC595B shift register), and a multi-channel switching circuit. The data slicer 130 may be, e.g., a comparator powered by the power management and storage circuit 120 and may convert the analog signal at the output of the rectifier 117 into digital pulses. The positive input of the data slicer 130 may be connected to the output of the rectifier 117, and the negative input of the data slicer 130 may be connected (i) to a reference, or “threshold” voltage (which may, e.g., be generated by a suitable voltage reference, or by a voltage divider connected, for example, to the output of the power management and storage circuit 120 (e.g., to the storage capacitor 125)), or (ii) to an adaptive reference voltage source such as the low-pass RC filter (including a series resistor 205 and a shunt capacitor 210) illustrated in FIG. 2. The time constant of this low-pass filter may be selected to be longer than the rise and fall times of the edges of the modulation waveform (discussed in further detail below).

The passive demodulator 135 may convert a pulse width modulated (PWM) signal into an amplitude-modulated signal. It may include (as shown) an RC circuit (including a resistor R1 and a capacitor C1) which accumulates a voltage that is approximately proportional to the width of each pulse of the PWM signal. The demodulator may also include a large resistor (R2) to discharge the accumulated voltage, and a diode to prevent the accumulated voltage from discharging back into the data slicer 130. In some embodiments the data slicer 130 has an output stage capable both of sourcing and sinking current, and the diode may be absent. The components may have respective values of (or within 50% of) the following: R1=1 kOhm, R2=15 kOhm, and C1=1.5 μF (with time constants, for example, of τ₁=R₁C₁=1.5e−3 and τ₂=R₂C₁=0.0225).

The inverter 145 (a NOT gate, e.g., a Texas Instruments SN74AUP2G14 low power inverter) may be used to invert the signal at the output of the slicer so as to provide, at the clock input of the shift register 140 (which may be rising-edge triggered), a series of rising edges, each corresponding to a falling edge in the modulation envelope of the control signal received from the external transmitter 110. If the shift register 140 clock input is falling-edge triggered, then the inverter 145 may be absent.

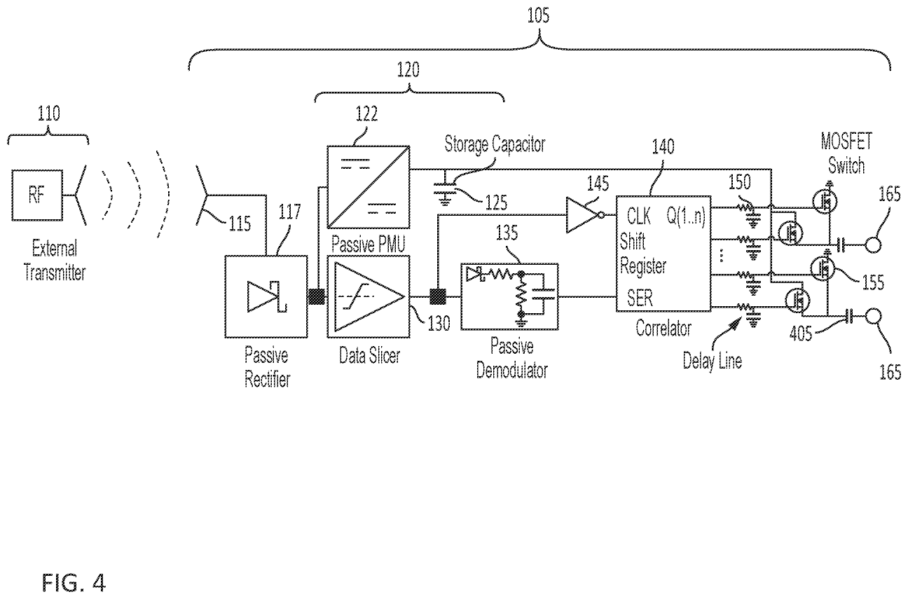

The shift register 140 may have a serial input (SER), a clock input (CLK), and a parallel output Q(1 . . . n). The shift register 140 may receive, at its serial input, the amplitude-modulated output of the demodulator, and, at each triggering clock edge, convert it into a digital bit (a zero or a one); these bits may then appear at the parallel output of the shift register 140. A channel may be activated when a one bit appears at the parallel output. In some embodiments, the input of a buffer (e.g., a Texas Instruments SN74AUP1G17 single Schmitt-trigger buffer) is connected to the output of the passive demodulator 135, and the output of the buffer is connected to the serial input of the shift register 140 (instead of the output of the passive demodulator 135 being connected directly to the serial input of the shift register 140, as illustrated in FIGS. 1 and 4).

In operation, if a long RF pulse is received by the RF front end 112, then a long DC pulse may be produced at the output of the rectifier 117. This long pulse may cause a fixed amplitude pulse of the same width to be produced at the output of the data slicer 130 (the amplitude being set by the power supply voltage supplied to the data slicer 130 by the power management and storage circuit 120). During the long pulse, the voltage at the output of the passive demodulator 135 may increase in accordance with the time constant corresponding to R1 and C1, and it may exceed the threshold for a high value at the serial input of the shift register 140 (the threshold for a high value being, e.g., if the shift register 140 is a complementary metal oxide semiconductor (CMOS) circuit, between 0.5 and 0.7 of the power supply voltage supplied to the shift register 140 by the power management and storage circuit 120). As such, the falling edge at the end of the long pulse, which may produce (via the inverter 145) a rising edge at the clock input of the shift register 140, may cause a one bit to be clocked into the shift register 140. If a short pulse is received by the RF front end 112, then the behavior of the circuit may be similar, except that the voltage at the output of the passive demodulator 135 may increase only to a voltage that does not exceed the threshold for a high value (and that may remain below the threshold for a low value (e.g., less than 0.3 of the power supply voltage supplied to the shift register 140 by the power management and storage circuit 120)), and as a result, the falling edge at the end of the short pulse may cause a zero bit to be clocked into the shift register 140. In this manner, an arbitrary n-bit data word (where n is the length of the shift register 140) may be stored in the shift register 140, by sending to the stimulator 105 a control signal including a corresponding sequence of long and short pulses.

Each of the outputs of the shift register 140 may be connected to a respective input of the multi-channel switching circuit, which, in the embodiment of FIG. 1, includes an array of delay lines 150 (e.g., RC low-pass filters, as shown) and a corresponding array of switches 155 (e.g., metal oxide semiconductor field effect transistor (MOSFET) switches (e.g., enhancement mode n-channel MOSFET switches, as illustrated). The delay line filters may have a sufficiently long time constant (e.g., longer than (e.g., ten times longer than) the time constant R₁C₁ of the passive demodulator 135) that when a data word is being loaded into the shift register, the passage of a one bit, which may temporarily make one of the outputs high, will not make the output high long enough to cause the corresponding switch to be turned on. In some embodiments, p-channel MOSFET switches (e.g., Nexperia PMZ320UPE p-channel MOSFET switches) are used instead of n-channel MOSFET switches, with corresponding changes in the control signals (e.g., with a value of zero in a shift register bit turning the corresponding switch on, and a value of one turning the switch off). In some embodiments, each of one or more of the outputs of the shift register 140 is connected directly to a respective stimulation electrode 165, and the shift register output supplies the stimulation current (which may be a few milliamperes, in some applications). In an embodiment in which each of the outputs of the shift register 140 is connected directly to a respective stimulation electrode 165, the delay lines 150 and the array of switches 155 may be absent.

In some embodiments, the circuit of FIG. 1 is used for sensing. In such an embodiment, each of the electrodes 165 may operate as a sensing electrode. Each switch 155, instead of connecting the electrode 165 to a DC power source, may, when closed, connect the corresponding electrode 165 to the antenna and to a nonlinear device (e.g., to one terminal of a varactor, the other terminal of which is connected to ground). The voltage applied to the nonlinear device (or the current driven through the nonlinear device) by the sensing electrode 165 may affect the nonlinear response of the nonlinear device to the RF signal from the antenna, and, accordingly, affect the amplitudes of one or more harmonics of the RF signal, generated by the nonlinear device, which may then be radiated by the antenna (which may be a broadband antenna, or a multi-band antenna (e.g., a dual-band antenna)). The voltage applied to the nonlinear device (or the current driven through the nonlinear device) by the sensing electrode 165 may then be inferred from the amplitudes (or relative amplitudes) of the one or more harmonics radiated by the antenna and detected outside of the body. In some embodiments, some of the channels of the implantable device are configured for, and used for, stimulating, and other channels of the implantable device are configured for, and used for, sensing.

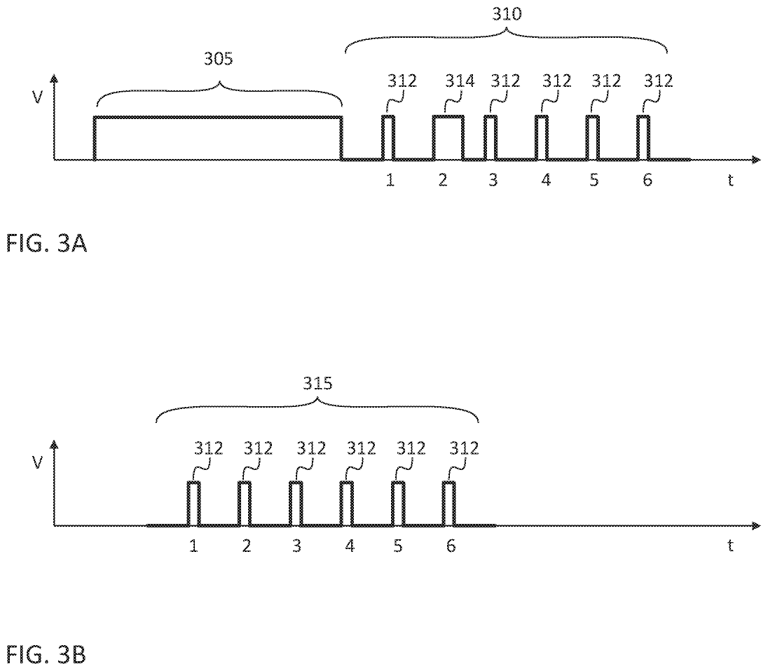

FIG. 3A shows an example of a modulation waveform that may be employed to charge the storage capacitor 125, and to program the shift register 140 with a (6-bit) data word, to turn on one of the channels (channel 2). A first long pulse (or “charging pulse”) 305 may be employed to charge the storage capacitor 125. A sequence of pulses 310, some of which may be short pulses 312 and some of which (e.g., one of which, as shown) may be long pulses 314, may then be used to load a data word into the shift register 140, with one or more one bits in the data word selecting channels to be turned on (causing current to be delivered to one or more corresponding stimulation electrodes 165 from the storage capacitor 125). The zero bits in the data word may cause the other channels to remain turned off. The example waveform of FIG. 3A may be used with a shift register 140 having a length of 6 bits, to turn on channel 2 (as a result of the waveform having a long pulse 314 in the corresponding position). FIG. 3B shows a waveform 315 (consisting of six short pulses 312) that may be used as a “reset” command, to turn off all six channels of the passive wireless multi-channel stimulator 105. Each of FIGS. 3A and 3B shows DC voltage (V) at the output of the data slicer 130 as a function of time (t).

In the example of FIG. 3A, channel 1 is not being turned on; if channel 1 were to be turned on, then the charging pulse 305 may be used for this purpose, and only five additional pulses may follow the charging pulse 305. FIGS. 3A and 3B are not drawn to scale. The edges of the waveforms are shown as vertical, for simplicity, in FIGS. 3A and 3B.

In some embodiments, each of the short pulses 312 has a length of no more than 0.6 the length of a long pulse 314; for example, each short pulse may have a length of about 3 ms and each long pulse may have a length of about 10 ms. The frequency at which stimulation pulses are applied may be greater than 1 Hz, and as high as, e.g., 50 Hz, with each cycle including (i) a charging pulse, (ii) a set of control pulses, to begin the delivery of the stimulation current, (iii) a time interval during which the stimulation current is delivered, and (iv) a reset signal.

For example, the charging pulse 305 may have a duration of between 200 ms and 1,000 ms. For an 8-bit shift register, the sequence of pulses 310 may include seven O-bits (short pulses 312) and one 1-bit (one long pulse 314) to form an 8-bit data word. With a separation time of at least 30 ms, the sequence of pulses 310 may last for 7×3+1×10+7×30=241 ms. The reset signal may be transmitted at a pulse width of 1 ms for a total duration of 8×1=8 ms.

The baseband circuit may, as explained above, extract the clock for the shift register from the control signal, thereby eliminating the need for an active local oscillator. In the embodiment of FIG. 1, the passive wireless multi-channel stimulator 105 contains only four components connected to a DC power source and relying on a power supply voltage for operation, namely, the converter circuit 122, the data slicer 130, the inverter 145, the and the shift register 140. These components may be obtained as off-the-shelf parts, or be integrated into an integrated circuit (e.g., a CMOS application specific integrated circuit (ASIC)) with extremely low current consumption. As a result, these components (and the entire passive wireless multi-channel stimulator 105) may be powered through the harvested RF energy which is available in the storage capacitor 125, and the passive wireless multi-channel stimulator 105 may be capable of operating as designed if no energy is stored in it (e.g., in the storage capacitor 125) at the time of implantation. The circuits of the passive wireless multi-channel stimulator 105 may turn off after stimulation, and remain in this state until the next stimulation.

FIG. 4 shows an example of circuit configuration for generating multichannel biphasic pulse stimulation. For each channel, two consecutive output bits of the shift register 140 may be used to control the positive and negative pulse delivery, respectively; each channel therefore uses two bits of the shift register. In each channel, a DC-blocking capacitor 405 is inserted before the stimulation electrode to accumulate charge for biphasic pulse stimulation. To generate a positive pulse in a channel, the shift register may output “10” in the corresponding two consecutive output bits. The digital “10” turns on the first transistor and turns off the second transistor, enabling electrical charges accumulated at the storage capacitor 125 to be delivered to the stimulation electrode. At the same time, the storage capacitor 125 also charges the DC-blocking capacitor to store energy. When the shift register receives a clock signal, the output may shift one bit, resulting in digital “01” being stored in the two consecutive output bits, turning off the first transistor and turning on the second transistor. The electrical charge stored in the DC-blocking capacitor may then discharge through the second transistor and generate a negative stimulation pulse at the electrode. Additional clock pulses may be applied to the shift register so that the shift register turns on the channels one at a time and produces a biphasic stimulation signal in each of the channels. In each of the embodiments of FIG. 1 and FIG. 4, one or more ground electrodes (not shown) may be used as a current return path from the tissue being stimulated.

The stimulator 105 may be compact, e.g., sufficiently small to fit within the intracranial space, for example. In some embodiments the stimulator 105 is fabricated on a substrate (e.g., by soldering components to the substrate) which may be a printed circuit board, e.g., a flex circuit fabricated on a flexible polyester film. In such an embodiment the overall thickness of the stimulator 105 may be approximately equal to the thickness of the substrate (which may have a thickness of between 0.1 mm and 1 mm) and the thickness of the thickest component (which may have a thickness of between 0.1 mm and 3 mm). The area of the circuit (excluding the wireless reception element 115) may be between 5 square millimeters and 300 square millimeters. In an embodiment in which some or most of the circuit elements are integrated into an ASIC, the stimulator 105 may be significantly smaller. In some embodiments, stimulation voltages of up to 8 volts may be achieved.

The rectifier 117 may provide a voltage sufficient for the power management and storage circuit 120 when the received power, at the output of the antenna, is between −1 dBm and −4 dBm. For example, the sensitivity (e.g., the minimum input voltage) of the power management and storage circuit 120 may be about 0.7 V, and the voltage provided by the rectifier 117 may be equal to 0.7 V for a received power of −3.5 dBm, for a first value of a matching inductor, e.g., 1.4 nH. As another example, the voltage provided by the rectifier 117 may be equal to 0.7 V for a received power of −1.5 dBm for a second value of the matching inductor, e.g., 4.7 nH. The matching inductor may be a series inductor. In some embodiments, the matching circuit includes an inductor connected in series with a resistor (connected in series with the antenna) or a suitable section of (e.g., microstrip) transmission line.

It will be understood that various variations in the embodiments (examples of which follow) may be practiced, to similar effect. In some embodiments, the number of channels may be increased by cascading several shift registers. In some embodiments, a separate capacitor, used to provide the stimulation current, may be charged by an additional converter circuit (separate from the converter circuit 122) that may also be connected to the rectifier 117 (in such an embodiment the storage capacitor 125 may power the data slicer 130, the inverter 145, and the shift register 140).

The signals at the inputs of the shift register 140 or at the outputs of the shift register 140 in the circuit of FIG. 1 may be used for various purposes instead of, or in addition to, the control of signals applied to stimulation electrodes 165. For example, the output of the inverter 145 and the output of the passive demodulator 135 may be (i) connected to a clock input and a serial input of a shift register in a second implanted device (which may share an enclosure with the portions of the circuit of FIG. 1 that are retained) and (ii) used to shift control values into the shift register, and thereby to control or program the second implanted device (e.g., to control the administered dose, if the second implanted device is a medication metering and administering device).

As another example, other circuits may be cascaded with the one or more cascaded shift registers. A digital potentiometer (cascaded with the shift register 140) having a wiper connected to the voltage control terminal of a second converter circuit (e.g., a Texas Instruments bq25570 DC-DC converter) for charging the capacitor may be used to limit the voltage on the storage capacitor 125 (and thereby to limit the magnitude of the stimulation). As another way to limit the voltage on the storage capacitor 125, the output of a serial DAC (cascaded with the shift register 140 and powered, e.g., by the power management and storage circuit 120), may be connected (directly, or through a buffer amplifier such as a source follower) to the storage capacitor 125, to charge the storage capacitor 125. In some embodiments, the converter circuit 122 is absent, and the storage capacitor 125 is charged directly (or through resistors or additional diodes) from the rectifier 117. In such an embodiment the magnitude and duration of the charging pulse 305 may be selected to charge the storage capacitor 125 with suitable voltages for powering the circuits of the stimulator 105, and for delivering an acceptable stimulation current, respectively.

As used herein, “a portion of” something means “at least some of” the thing, and as such may mean less than all of, or all of, the thing. As such, “a portion of” a thing includes the entire thing as a special case, i.e., the entire thing is an example of a portion of the thing. As used herein, when a second quantity is “within Y” of a first quantity X, it means that the second quantity is at least X-Y and the second quantity is at most X+Y. As used herein, when a second number is “within Y %” of a first number, it means that the second number is at least (1-Y/100) times the first number and the second number is at most (1+Y/100) times the first number. As used herein, the word “or” is inclusive, so that, for example, “A or B” means any one of (i) A, (ii) B, and (iii) A and B.

As used herein, when a method (e.g., an adjustment) or a first quantity (e.g., a first variable) is referred to as being “based on” a second quantity (e.g., a second variable) it means that the second quantity is an input to the method or influences the first quantity, e.g., the second quantity may be an input (e.g., the only input, or one of several inputs) to a function that calculates the first quantity, or the first quantity may be equal to the second quantity, or the first quantity may be the same as (e.g., stored at the same location or locations in memory as) the second quantity. As used herein, when a circuit is described as being “for” performing a function, it means that the circuit is capable of performing the function; the circuit may also be capable of performing other functions.

The terminology used herein is for the purpose of describing particular embodiments only and is not intended to be limiting of the inventive concept. As used herein, the terms “substantially,” “about,” and similar terms are used as terms of approximation and not as terms of degree, and are intended to account for the inherent deviations in measured or calculated values that would be recognized by those of ordinary skill in the art.

Any numerical range recited herein is intended to include all sub-ranges of the same numerical precision subsumed within the recited range. For example, a range of “1.0 to 10.0” or “between 1.0 and 10.0” is intended to include all subranges between (and including) the recited minimum value of 1.0 and the recited maximum value of 10.0, that is, having a minimum value equal to or greater than 1.0 and a maximum value equal to or less than 10.0, such as, for example, 2.4 to 7.6. Similarly, a range described as “within 35% of 10” is intended to include all subranges between (and including) the recited minimum value of 6.5 (i.e., (1-35/100) times 10) and the recited maximum value of 13.5 (i.e., (1+35/100) times 10), that is, having a minimum value equal to or greater than 6.5 and a maximum value equal to or less than 13.5, such as, for example, 7.4 to 10.6. Any maximum numerical limitation recited herein is intended to include all lower numerical limitations subsumed therein and any minimum numerical limitation recited in this specification is intended to include all higher numerical limitations subsumed therein.

It will be understood that when an element is referred to as being “immediately connected” or “immediately coupled” to another element, there are no intervening elements present. As used herein, “connected” means connected by a signal path (e.g., a conductor or a waveguide) that may contain arbitrary intervening elements, including intervening elements the presence of which qualitatively changes the behavior of the circuit. As used herein, “directly connected” means (i) “immediately connected” or (ii) connected with intervening elements, the intervening elements being ones (e.g., low-value resistors or inductors, short sections of transmission line, or short sections of waveguide) that do not qualitatively affect the behavior of the circuit. When a first element is directly connected to a second element, which in turn is directly connected to a third element, the first element may be said to be connected to the third element “through” the second element.

Although exemplary embodiments of a passive wireless multi-channel stimulator have been specifically described and illustrated herein, many modifications and variations will be apparent to those skilled in the art. Accordingly, it is to be understood that a passive wireless multi-channel stimulator constructed according to principles of this disclosure may be embodied other than as specifically described herein. The invention is also defined in the following claims, and equivalents thereof.

What is claimed is:

1. A system, comprising:
a wireless reception element, for receiving power wirelessly through tissue of a subject;
a power management and storage circuit, for storing a portion of the received power; and
a control circuit for controlling the delivery of current from the power management and storage circuit to each of a plurality of stimulation electrodes individually, based on a modulation of the received power.

2. The system of claim 1, wherein the wireless reception element is an antenna.

3. The system of claim 1, wherein the control circuit is configured:
to receive a first pulse having a first pulse width and a second pulse having a second pulse width, different from the first pulse width,
to convert the first pulse to a first binary value; and
to convert the second pulse to a second binary value different from the first binary value.

4. The system of claim 3, comprising a rectifier for rectifying an alternating current (AC) signal received from the wireless reception element.

5. The system of claim 4, wherein the control circuit comprises a passive demodulator circuit for converting the first pulse and the second pulse to the first binary value and the second binary value, respectively.

6. The system of claim 5, wherein the passive demodulator circuit comprises a low-pass filter.

7. The system of claim 6, wherein the low-pass filter is a resistor-capacitor (RC) low-pass filter.

8. The system of claim 3, wherein the control circuit comprises a shift register, configured:
to receive the first binary value and the second binary value;
to output the first binary value at a first parallel output of the shift register; and
to output the second binary value at a second parallel output of the shift register.

9. The system of claim 8, comprising a rectifier for rectifying an alternating current (AC) signal received from the wireless reception element,
wherein:
the control circuit comprises a rectifier for rectifying an alternating current (AC) signal received from the wireless reception element; and
the shift register has a clock input, connected to the rectifier.

10. The system of claim 9, wherein the clock input is connected to the rectifier through an inverter.

11. The system of claim 10, wherein:
the first parallel output of the shift register is connected to a first stimulation electrode of the plurality of stimulation electrodes, and
the second parallel output of the shift register is connected to a second stimulation electrode of the plurality of stimulation electrodes.

12. The system of claim 11, wherein the first parallel output of the shift register is connected to the first stimulation electrode of the plurality of stimulation electrodes through a switch, the switch being configured to selectively route current from the power management and storage circuit to the first stimulation electrode, in accordance with a control signal received from the first parallel output of the shift register.

13. The system of claim 12, wherein the first parallel output of the shift register is connected to the switch through a low-pass filter.

14. The system of claim 1, wherein the power management and storage circuit comprises a first capacitor for supplying the current for the stimulation electrodes.

15. The system of claim 14, wherein the power management and storage circuit comprises a direct current to direct current (DC-DC) converter, configured to charge the first capacitor.

16. The system of claim 15, wherein the control circuit comprises a shift register, configured to be powered by the DC-DC converter and the first capacitor.

17. The system of claim 16, further comprising a rectifier and a data slicer, wherein:
the control circuit comprises a passive demodulator circuit;
the rectifier is connected to the wireless reception element; and
the data slicer has an input connected to the rectifier, an output connected to the passive demodulator circuit, and a power supply connection connected to the first capacitor.

18. The system of claim 17, comprising a dielectric or semiconductor substrate supporting the power management and storage circuit and the control circuit, the substrate, the power management and storage circuit and the control circuit together occupying a volume of less than 1,000 cubic millimeters.

19. The system of claim 18, wherein the substrate is substantially flat, and has a thickness of less than 4 mm.

20. A system, comprising:
a wireless reception element, for receiving power wirelessly through tissue of a subject; and
a shift register,
the shift register being configured:
to be powered by the wirelessly received power;
to have stored in it a data word received as a modulation of the wirelessly received power; and
to control delivery of current to a stimulation electrode, of a plurality of stimulation electrodes, based on a bit of the data word.
